(19) United States
(12) Patent Application Publication
(10) Pub. No.: US 2017/0065448 A1
(43) Pub. Date: Mar. 9, 2017

(54) BRACE FOR PROTECTING A REGION OF A LIMB

(71) Applicant: Andrew MICHELL, Cambridge (GB)

(72) Inventor: Andrew MICHELL, Cambridge (GB)

(21) Appl. No.: 15/120,449

(22) PCT Filed: Feb. 19, 2015
(86) PCT No.: PCT/GB2015/050486

§ 371 Date: Aug. 19, 2016

(30) Foreign Application Priority Data
Feb. 19, 2014 (GB) 1402953.2

(51) Int. Cl. A61F 5/01

(52) U.S. Cl. CPC A61F 5/0118; A61F 5/013; A61F 2005/0165

(57) ABSTRACT

A medical or surgical brace apparatus (100) for treating a neuropathy and protecting the course of a nerve in a limb of the body. The apparatus comprises a sleeve baselayer (1) formed to fit, or configured to conform to fit, a portion of the limb around a joint and to grip the limb, such that a portion of the sleeve baselayer is proximal to the joint and a portion is distal of the joint; and a deformable exoskeleton (3) configured to fit around the joint of a body such that a portion of the exoskeleton is proximal to the joint and a portion is distal of the joint, the exoskeleton comprising a spine (10) and a plurality of ribs (9) coupled to the spine; wherein the exoskeleton is coupled to the sleeve baselayer such that when the sleeve baselayer is worn fitted on the limb, the spine of the exoskeleton is aligned with, and arranged to follow, at least part of the course of the nerve, and the ribs are aligned transverse to the course of the nerve to bridge across the nerve to protect it.

### FIELD OF THE INVENTION

The present disclosure relates to a brace for protecting a region of a limb and an apparatus for inhibiting the range of movement of a joint of the body.

### BACKGROUND

Ulnar neuropathy is the second most common entrapment neuropathy after carpal tunnel syndrome. The symptoms of tingling/numbness affecting the ulnar side of the hand are even more common still. For carpal tunnel syndrome the established first line treatment, proven effective in clinical trials, is wrist splinting. The current Cochrane review of evidence for treatment of ulnar neuropathies highlights the clinical need for conservative treatments for ulnar symptoms, such as splints.

At present, patients presenting to their doctor with symptoms of a focal neuropathy, for example an ulnar neuropathy, are advised to (a) try and avoid direct pressure on the ulnar nerve at the elbow, the most common site of entrapment, and (b) avoid prolonged extreme flexion of the elbow beyond 90 degrees, especially at night, since this causes tension and pressure on the nerve.

### SUMMARY OF THE INVENTION

In one aspect there is provided a medical or surgical brace apparatus for treating a neuropathy and protecting the course of a nerve in a limb of the body, comprising:
a sleeve baselayer formed to or configured to conform to a portion of the limb around a joint and to grip the limb, such that a portion of the sleeve baselayer is proximal to the joint and a portion is distal of the joint; and
a deformable exoskeleton configured to fit around the joint of a body such that a portion of the exoskeleton is proximal to the joint and a portion is distal of the joint, the exoskeleton comprising a spine aligned with the course of the nerve and a plurality of ribs coupled to the spine and extending transverse to the course of the nerve; wherein
the exoskeleton is coupled to the sleeve baselayer such that when the sleeve baselayer is worn fitted on the limb, the exoskeleton is aligned with, and arranged to follow at least part of the course of the nerve to bridge across the nerve to protect it.

The apparatus may be configured to treat an ulnar neuropathy, and the sleeve baselayer and exoskeleton are configured to fit around an elbow joint.

The apparatus may comprise a flexible strap configured to be couplable to the baselayer to inhibit movement of the joint outside a selected range more than within the selected range. The exoskeleton may comprise a guide to limit movement of the strap in a direction transverse to the longitudinal axis of the limb.

In another aspect there is provided brace apparatus for protecting the course of a nerve in a limb of the body, comprising:
a baselayer; and
a deformable exoskeleton;
wherein the baselayer is formed to fit the limb; and
the exoskeleton is coupled to the baselayer such that when the baselayer is worn fitted on the limb, the exoskeleton is aligned with, and arranged to follow at least a part of the course of the nerve and to bridge across the nerve to protect it.

The baselayer may be formed to fit the elongate region of the limb of the body. The exoskeleton may be arranged to bridge across the nerve at least twice. The exoskeleton may be arranged to bridge across the nerve at at least two separate places along its course. The exoskeleton may be arranged to follow the course of the nerve close to a joint of the body, for example the elbow joint. The exoskeleton may comprise an elongate spine arranged so that, when the base layer is worn fitted to the limb, the course of the spine corresponds to the course of the nerve.

In another aspect there is provided a brace for protecting a region of a limb of the body, comprising:
a baselayer; and
a deformable exoskeleton; wherein
the baselayer is formed to fit the limb; and
the exoskeleton is coupled to the baselayer such that when the baselayer is worn on the limb the exoskeleton is arranged to bridge across the region of the limb at at least two separate places.

The region of the limb may be elongate, and may comprise a nerve, for example the ulnar nerve or the peroneal nerve. The region of the limb may correspond to the course of a nerve along the limb.

The baselayer may comprise a sleeve adapted to fit to a first portion of a limb, proximal to a joint, and a second part of the limb, distal to the joint, wherein the region of the limb, or the course of the nerve, extends across the joint.

The exoskeleton may bridge across the region of the limb, such that upon the application of pressure to the exoskeleton, pressure is distributed away from the region of the limb or nerve, for example to either side (medial or lateral) of the course of the nerve.

Advantageously the above aspects provide a cheap and lightweight single patient use solution which protects, for example, the ulnar nerve from direct external pressure by use of the deformable exoskeleton. Because the exoskeleton bridges the nerve, pressure over the ulnar nerve at the elbow, for example, is redirected away from the nerve.

The exoskeleton may comprise at least two ribs. The at least two ribs may be arranged so that each rib bridges across the region of the limb or nerve, so that the exoskeleton bridges across the region of the limb at least twice. The at least two ribs may be coupled together at their ends. The ribs may be coupled to form a meandering pattern, for example a zig-zag or square-wave shape. The meandering pattern may be meandering with respect to the region of the limb, for example the meandering pattern may meander over the course of a nerve along the limb.

The exoskeleton may comprise a spine. The spine may be aligned with the course of a nerve running in the limb. The spine may couple the ribs. The ribs may be coupled by a hinge, for example a living hinge. The use of the ribs allows the exoskeleton to be lightweight and breathable.

The exoskeleton may be resiliently deformable. The exoskeleton may be made from a polymer, for example a plastics material. The exoskeleton may comprise foam.

The baselayer may be supportive and breathable, and may be a padded lining. The baselayer may be a mesh. The baselayer may be a fabric, for example neoprene. The baselayer may be a spacer fabric. The baselayer may be configured to wick away sweat. The baselayer may comprise a dressing for contact with, for example, a wound. The exoskeleton may be coupled to the baselayer by, for example, fusing the exoskeleton to the baselayer or stitching the exoskeleton to the baselayer. The exoskeleton may be integrated into the baselayer. The exoskeleton may be removable from the baselayer.

The brace may be adapted to protect a nerve at a joint of a body, for example the elbow joint or knee joint. The brace may be adapted to fit around a joint of the body, for example the elbow joint, the knee joint, the shoulder, the wrist or the ankle. The brace may be adapted to treat a focal neuropathy, for example ulnar neuropathy or peroneal neuropathy. The exoskeleton may be adapted to fit around a joint of the body, for example the olecranon or patella. The exoskeleton may have a hole configured to fit around a bony prominence at a joint, for example the olecranon or patella. Advantageously this may keep the brace stable when the joint is moved.

Another aspect provides an apparatus for inhibiting the range of movement of a joint of the body, comprising:
a sleeve adapted to wrap around a limb either side of the joint of the body, wherein the sleeve provides two anchors, one located either side of the joint; and
a flexible strap configured to be couplable to the two anchors;
such that when coupled to the anchors, the strap allows a selected range of movement of the joint, but inhibits movement of the joint outside the selected range.

Advantageously the apparatus provides a safe, cheap and easy to use apparatus that may be easily engaged by a user to restrict movement of, for example, their elbow joint at night. The apparatus may be easily disengaged to allow free movement of the joint.

The apparatus may comprise an exoskeleton coupled to the sleeve such that when the sleeve is worn on the limb, it is arranged to cross the region of the limb at least twice.

The apparatus may comprise a flexible strap configured to be couplable to the baselayer to inhibit movement of the joint outside a selected range more than within a selected range. The strap may be extendible. The strap may comprise a biasing means to inhibit extension of the strap. The strap may have a fixed maximum length.

The strap may comprise a reattachable fastener. The reattachable fastener may be releasable such that the strap no longer inhibits the range of movement of the joint. The fastener may be arranged to couple the strap to at least one of the anchors.

The strap may comprise a core arranged to be slidable within a tube within a range corresponding to the selected range of movement of the joint. The tube may be configured to be coupled to one anchor and the core may be configured to couple to the other anchor.

The tube may comprise a channel within which the core runs. The tube may comprise a plurality of slits in one side to allow flexure of the tube without constricting the width of the channel of the tube. The core and the tube may have rectangular cross-section, or any other regular or irregular cross-section.

The core may comprise a stop, and the tube may comprise one or more retaining members, such that the range of movement of the core within the channel of the tube, and hence the selected range of movement, can be adjusted. The retaining members may be adapted to engage with the stop of the core.

The apparatus may comprise a movement detector to detect extension of the strap. The movement detector may detect strain on the strap. The movement detector may detect a degree of movement of the core within the tube.

The apparatus may comprise a guide to limit movement of the strap in a direction transverse to the joint. The guide may be adapted to position at least a portion of the strap over the bony prominence at the outer edge of the joint. The guide may be coupled to the exoskeleton.

In another aspect there is provided a kit of parts for a medical or surgical brace apparatus for treating a neuropathy and protecting the course of a nerve in a limb of the body, comprising:
sleeve baselayer formed to or configured to conform to a portion of the limb around a joint and to grip the limb, such that a portion of the sleeve baselayer is proximal to the joint and a portion is distal of the joint; and
a deformable exoskeleton configured to fit around the joint of a body such that a portion of the exoskeleton is proximal to the joint and a portion is distal of the joint, the exoskeleton comprising a spine aligned with the course of the nerve and a plurality of ribs coupled to the spine and extending transverse to the course of the nerve; wherein
the exoskeleton is couplable to the sleeve baselayer such that when the sleeve baselayer is worn fitted on the limb and the exoskeleton is coupled to the sleeve baselayer, the exoskeleton is aligned with, and arranged to follow at least part of the course of the nerve to bridge across the nerve to protect it.

The brace of one aspect may be combined with the apparatus of the other aspect.

### DRAWINGS

Embodiments of the disclosure will now be described, by way of example only, with reference to the accompanying drawings, in which:

FIG. 1 shows a perspective view of a brace comprising an apparatus for inhibiting the range of movement of a joint;

FIG. 2 shows a perspective view of an exoskeleton for use in the brace of FIG. 1 having a guide for guiding the strap;

FIG. 3 shows a cross-section of a baselayer for use with the brace of FIG. 1;

FIG. 4a shows a cross-section through an arm showing the site of common ulnar neuropathy;

FIG. 4b shows perspective views of an exoskeleton for use with the brace of FIG. 1 bridging a nerve;

FIG. 5 shows a cross-section through an arm showing the pivot point of the elbow joint;

FIGS. 6a and 6b show a cross-section of a strap for use with an apparatus shown in FIG. 1 in two positions;

FIGS. 7a and 7b show a cross-section of a strap for use with an apparatus shown in FIG. 1 in two positions;

FIGS. 8a, 8b and 8c show cross-sections of a strap for use with an apparatus shown in FIG. 1;

FIGS. 9a and 9b show a cross-section of a strap for use with an apparatus shown in FIG. 1 in two positions;

FIGS. 10a, 10b and 10c show perspective views of a strap for use with an apparatus shown in FIG. 1;

FIG. 11a shows a perspective view of part of a strap for use with an apparatus shown in FIG. 1;

FIGS. 11b and 11c show cross-sections of the part of the strap shown in FIG. 11a;

FIG. 12 shows another configuration of a brace for protecting a nerve of the body;

FIG. 13 shows another configuration of an exoskeleton for a brace for protecting a nerve of the body;

FIG. 14 shows a perspective view of a resilient member for use with the brace of FIG. 1.

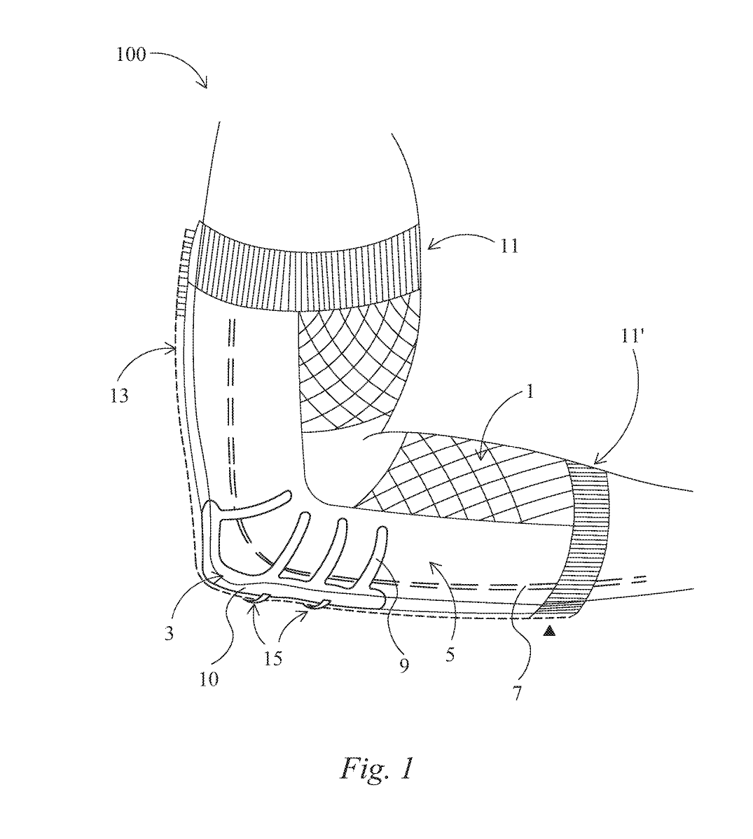

FIG. 1 shows a brace 100 comprising a sleeve of baselayer 1 formed to fit an arm 5 around the elbow joint. The baselayer 1 comprises a compressible spacer fabric and a breathable fabric. The sleeve of baselayer 1 comprises a cuff at each end that provides an anchor 11, 11′.

The brace 100 comprises a deformable exoskeleton 3 coupled to the sleeve of baselayer 1 and adapted to fit around the elbow joint at the medial epicondule 300 when the sleeve is worn on an arm 5 of a wearer. The exoskeleton 3 comprises four ribs 9 connected by a spine 10. The sleeve is arranged so that three of the ribs 9 are distal of the elbow joint and one of the ribs 9 is proximal to the elbow joint. When the sleeve is worn on the arm 5 of a wearer, the spine 10 is aligned with the course of the nerve 7 and is curved around the elbow joint. In this way, the exoskeleton 3 bridges the nerve 7 so that a portion of the exoskeleton 3 is medial of the nerve 7 and a portion of the exoskeleton 3 is lateral of the nerve 7.

The brace 100 further comprises a strap 13 that is coupled to the two anchors 11, 11′.

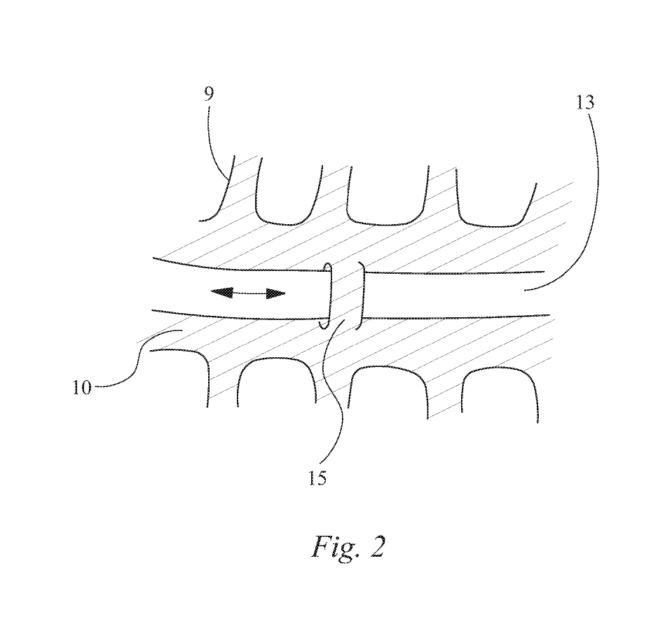

The strap 13 runs along the outside of the baselayer 1 and over the exoskeleton 3. The exoskeleton 3 comprises two loops that act as guides 15 for the strap 13. An example of this can also be seen in FIG. 2.

The cuffs of the baselayer 1 shown in FIG. 1 are adapted to grip the arm 5 tightly to provide anchors 11, 11′. The brace 100 comprising the strap 13 is configurable to allow a selected range of free movement of the joint, but to inhibit movement of the joint outside this selected range. The exoskeleton 3 has a degree of flexibility such that it is operable to deform with movement of the elbow joint, yet still provide protection to the underlying arm 5 and particularly to the cubital tunnel and region between the olecranon and medial epicondule 300.

The strap 13 is flexible and adapted to bend with flexion or extension of the joint. The guides 15 are operable to limit movement of the strap 13 transverse to the joint. An example of this is shown in FIG. 2.

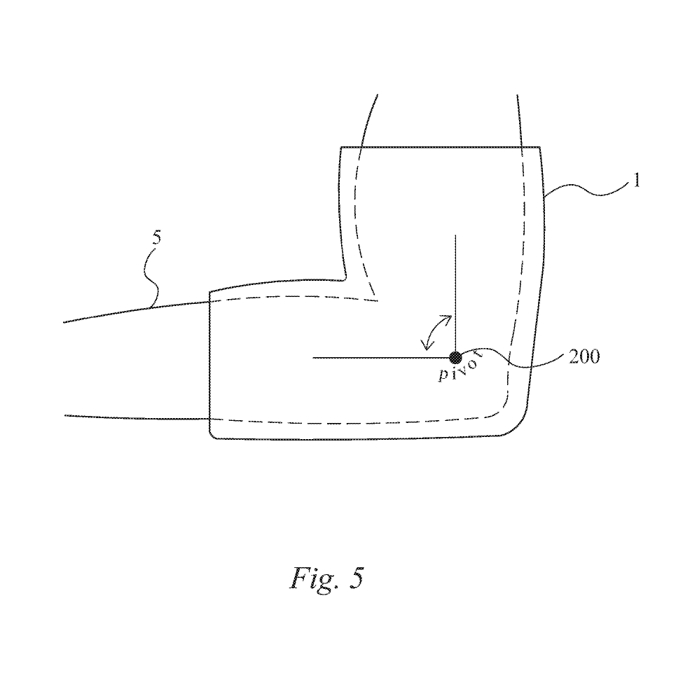

The elbow joint flexes and extends about a pivot point 200, for example as shown in FIG. 5. As the wearer's arm 5 is flexed or extended, the strap 13 slides over the exoskeleton 3 and is guided by the guides 15, as shown in FIG. 2.

In use when worn by a wearer, the brace 100 shown in FIG. 1 inhibits movement of a wearer's elbow joint past a selected range. In addition, the brace 100 reduces pressure on the ulnar nerve 7, for example when contact is made between the elbow joint and a surface.

In use when worn by a wearer, the cuffs of the baselayer 1 of the brace 100 firmly fix the brace 100 in place on the arm 5 and over the elbow joint, and provide two anchors 11, 11′. When worn, the exoskeleton 3 centres over the olecranon 400. The exoskeleton 3 reduces direct pressure on the ulnar nerve 7 because the ribs 9 bridge across the nerve 7 at a number of points. The baselayer 1 also absorbs pressure and deformation of the exoskeleton 3.

In use when worn by a wearer, the strap 13 inhibits the range of movement of the joint, and hence flexion and extension of the joint, if the range of movement is outside a selected range. The strap 13 inhibits the range of movement of the joint by providing a force, for example a pulling force, on the two anchors 11, 11′ when the range of movement is outside the selected range.

It may be desirable at some times, for example mealtimes, to allow extreme elbow flexion beyond the selected range without removing the brace 100 from the wearer's arm 5. To do this, one of the reattachable fasteners 27, 29 is detached. As well as removing the inhibition of the movement of the joint, detaching one of the reattachable fasteners 27, 29 also allows the selected range of movement of the joint to be controlled.

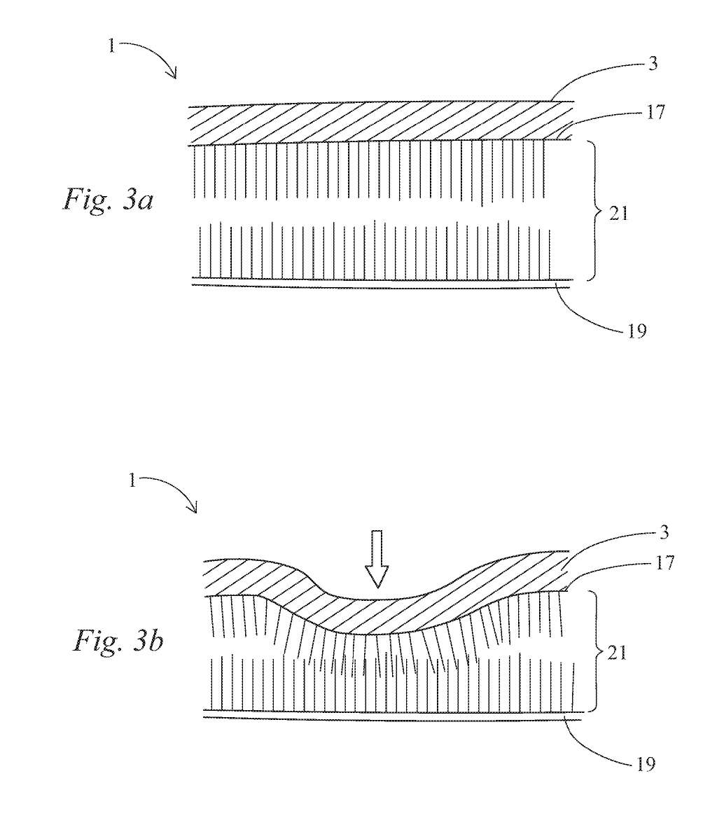

FIGS. 3a and 3b show an example baselayer 1 for use with the brace 100 of FIG. 1. The example baselayer 1 is a breathable, lightweight spacer material obtainable from Baltex®. The baselayer 1 comprises two layers 17, 19 with a mesh network in-between the two layers 17, 19. The mesh network comprises a plurality of opposing filaments 21 transverse to the two layers 17, 19.

FIGS. 3a and 3b illustrate a baselayer 1 that is configured to be deformable. For the baselayer 1 shown in FIG. 3, as pressure is applied to the exoskeleton 3, and therefore to the outer layer 17 of the baselayer 1, the filaments 21 between the outer layer 17 and the inner layer 19 mesh and become intertwined or flex. Therefore the baselayer 1 is operable to absorb a degree of deformation of the exoskeleton 3.

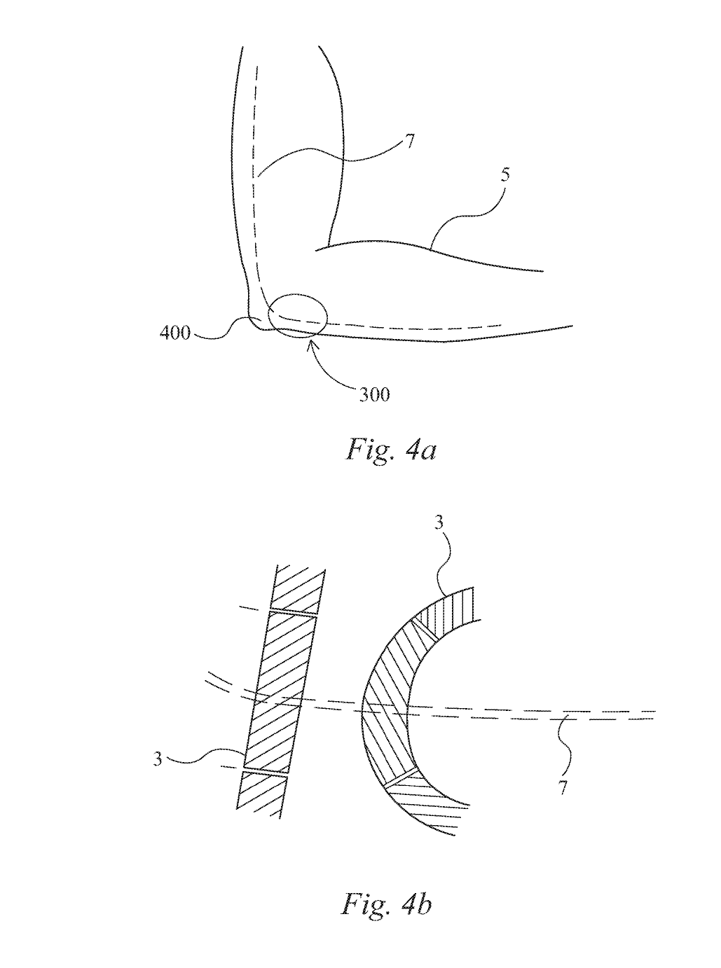

FIG. 4a shows an elbow joint with the site of the medial epicondule 300 and the olecranon 400. FIG. 4b shows an example of an exoskeleton 3. The ribs 9 of the exoskeleton 3 are arranged to grip a portion of the arm 5 such that the ribs 9 are arranged transverse to the ulnar nerve 7 and provide a bridge to the ulnar nerve 7 when the sleeve is worn on an arm 5 of a wearer.

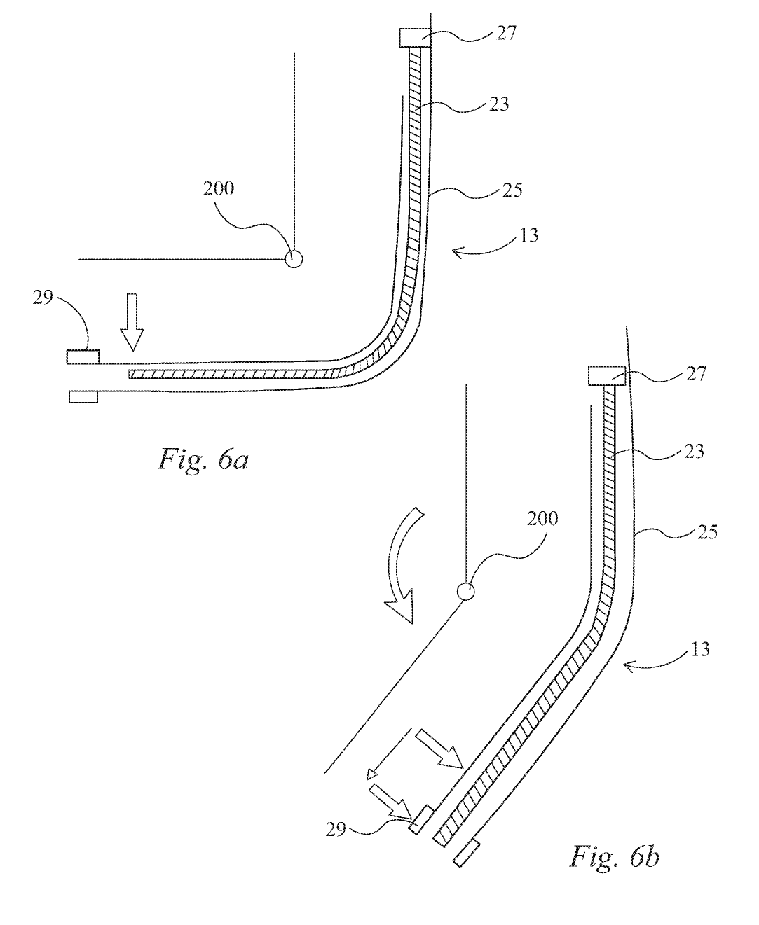

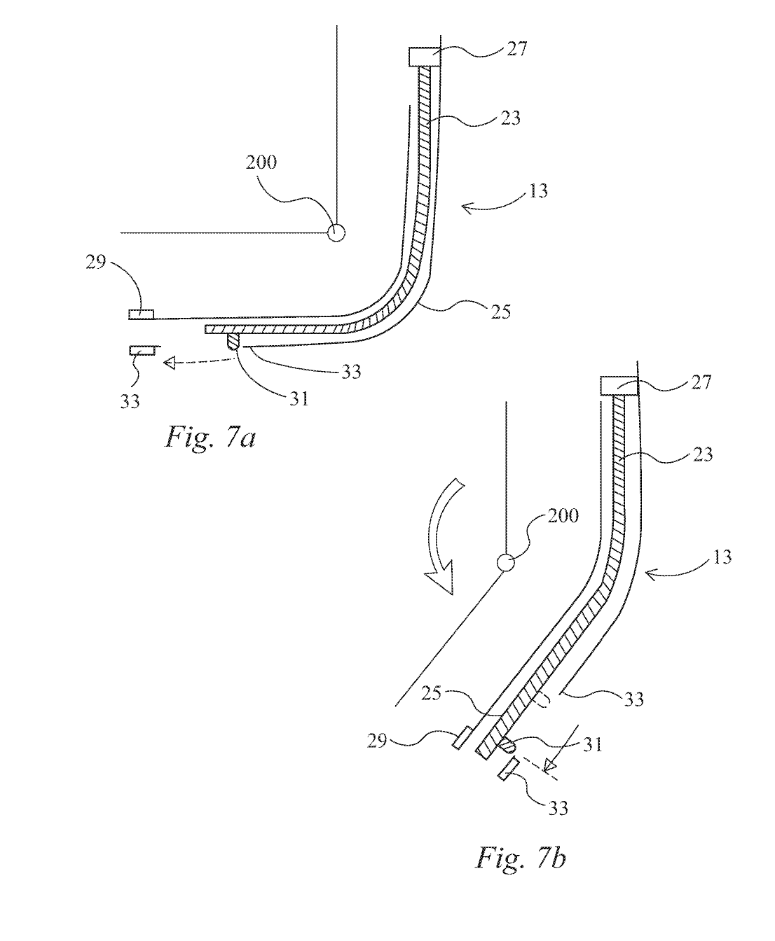

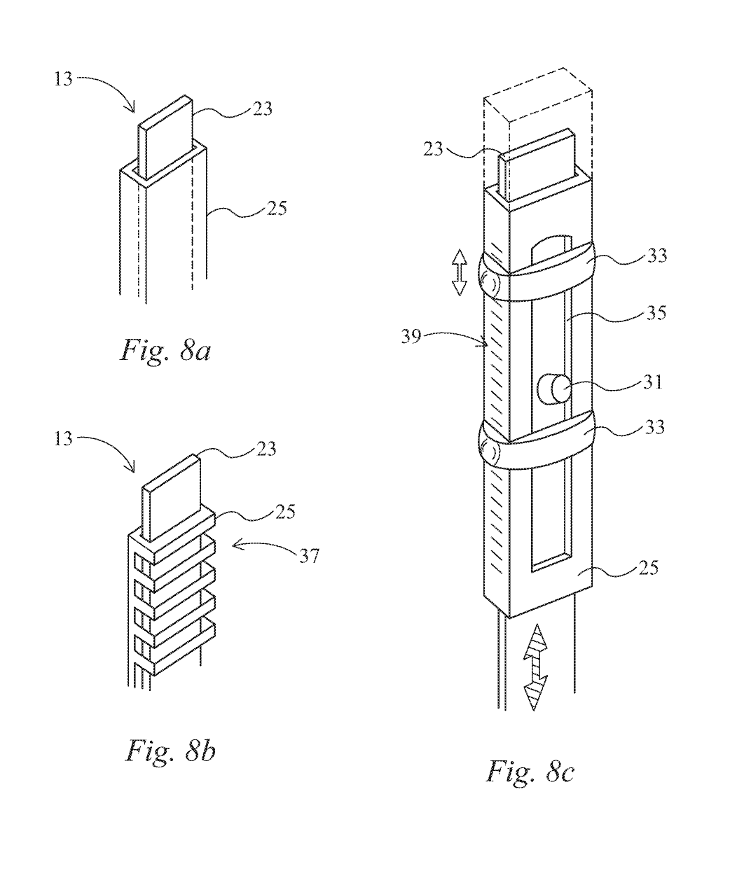

FIGS. 6a to 8c show an example of a strap 13 suitable for use with the brace 100 of FIG. 1. The strap 13 comprises a core 23 running within a tube 25. As shown in FIG. 8a, the core 23 has a rectangular cross-section. The tube 25 comprises a channel 35 with a complementary cross-section to fit the core 23, so that the core 23 runs within the channel 35 of the tube 25. The core 23 is coupled to one anchor 11 through a fastener 27 at one end, and the tube 25 is coupled to another anchor 11′ through another fastener 29 at the other end.

As shown in FIGS. 6a to 8c, the core 23 of the example strap 13 comprises a stop 31 and a pair of retaining members 33. The distance between the retaining members 33 defines a length corresponding to the range of movement of the core 23 within the channel 35 of the tube 25, which corresponds to the selected range of movement of the joint. As shown in FIG. 8c, the retaining members 33 are straps that run over the channel 35 of the tube 25, and the stop 31 comprises a projection from the core 23 that is adapted to engage with the retaining members 33. The stop 31 is cylindrical with a circular cross-section. The retaining members 33 are adapted to bridge the channel 35 and are approximately U-shaped in cross-section, having a cross-section adapted to fit over a portion of the tube 25. The tube 25 has a plurality of complementary notches 39 running along two opposite sides of the tube 25 to couple with the retaining members 33. As shown in FIG. 8b, the tube 25 has a plurality of slits 37 along one side. The slits 37 extend the entire width of the side of the tube 25 and are separated by ridges of approximately equal breadth to the breadth of the slits 37.

For the example strap 13 shown in FIGS. 6a to 8c, the core 23 and the tube 25 are flexible, and the core 23 is slidable within the channel 35 of the tube 25. The retaining members 33 are couplable to the tube 25 of strap 13 by way of an interference fit such that the retaining members 33 can clip onto the tube 25. The retaining members 35 are detachable and reattachable to the tube 25 of the strap 13 such that the uninhibited length of movement of the core 23 within the channel 35 of the tube 25 and hence the corresponding uninhibited selected range of movement of the joint is adjustable. The plurality of slits 37 along one edge of the tube 25 allow the strap 13 to bend without the channel 35 of the tube 25 being constricted.

For the example strap 13 shown in FIGS. 6a to 8c, once the strap 13 is fastened to the brace 100, one way of controlling the selected range of movement of the joint is to adjust the separation of the retaining members 33 on the tube 25 of the strap 13. When the joint flexes or extends, the core 23 slides within the channel 35 of the tube 25 and the retaining members 33 can be adjusted so that they engage with the stop 31 to prevent the core 23 from sliding any further within the channel 35 of the tube 25 in that direction.

In some configurations, the brace 100 and the baselayer 1 is formed to fit another limb, for example a leg, for example around the knee joint. In some configurations the brace 100 is adapted for protecting wounds or other nerves within the body. The brace may take any other shape, regular or irregular.

The baselayer 1 may have only one anchor 11 or may have more than two anchors 11, 11′. The cuffs at each end of the sleeve of the baselayer 1 may be adjustable, for example the cuffs may comprise adjustable straps which may comprise hook and loop material, or any other means to adjustably, detachably and reattachably fasten the baselayer 1 to the body, for example a strap and buckle, buttons or poppers. In this way, the fit of the anchors 11, 11′ and the baselayer 1, and hence the brace 100 as a whole, is adjustable. In some configurations the cuffs of the sleeve of the baselayer 1 comprise a biasing means to grip the limb, for example, the cuffs may comprise elastic, or the baselayer 1 may comprise a resiliently deformable member such that the cuffs of the sleeve of the baselayer are biased to grip the limb. In some configurations the baselayer 1 comprises a different material, for example the baselayer 1 may comprise neoprene®.

The exoskeleton 3 may be detachable and reattachable to the baselayer 1. Alternatively, the exoskeleton 3 may be fused to or integrally moulded with the baselayer 1. The exoskeleton 3 may be coupled to the baselayer 1 by other means, for example, the exoskeleton 3 may be coupled to the baselayer 1 by stitching, by buttons, by hook and loop material or by poppers. The exoskeleton 3 may comprise foam and/or a polymer, for example polyethylene, for example high density polyethylene (HDPE). In some configurations, the exoskeleton 3 is less than 3 mm thick, for example less than 2.5 mm thick, for example 2 mm thick.

In some configurations, if required, the exoskeleton 3 may be moulded to better fit the user, for example the user's joint, for example the user's elbow joint. The exoskeleton 3 may be moulded in situ whilst worn by a user, or prior to being worn by a user.

The exoskeleton 3 may comprise more or less than four ribs 9. The ribs 9 may all be configured to be one side of the joint, for example proximal or distal of the joint, or may be equally distributed either side of the joint, for example equally distributed proximally and distally of the joint. In some configurations the ribs 9 are parallel to each other and transverse to the region of the limb. In some configurations the ribs 9 are not parallel, for example the ribs 9 may be at an angle to each other, for example between 5 and 175 degrees, for example between 5 and 85 degrees, for example 45 degrees. The ribs 9 may be coupled at their ends. The ribs 9 may form a meandering pattern, for example, the ribs 9 may form a square-wave or zig-zag pattern, such that the exoskeleton 3 crosses the region of the limb at least twice. For example, the exoskeleton 3 may form a zig-zag or square wave pattern across the region of the limb.

In some configurations, the exoskeleton 3 may comprise a single rib 9. For example, the exoskeleton 3 may comprise a meandering ridge or pattern that meanders across a region of a limb of the body, such that the ridge crosses the region of the limb at least twice. For example, the exoskeleton 3 may form a zig-zag or square wave pattern across the region of the limb.

The exoskeleton 3 may comprise a spine 10. In some configurations the exoskeleton 3 comprises at least two spines 10. The spine(s) 10 may be aligned with a portion of the limb, for example a nerve in the limb. The spine(s) 10 may couple to the exoskeleton 3 at any point on the exoskeleton 3. For example, if there are at least two ribs 9, the spine(s) 10 may couple to the ribs 9 at either end of the ribs 9 or anywhere in the middle of the ribs 9.

In some configurations the exoskeleton 3 comprises a hole or indentation in the spine 10 so that the exoskeleton 3 is adapted to fit over a part of the body, for example a bone, for example a bony prominence, for example the olecranon 400 or patella. The hole in the spine 10 of the exoskeleton 3 may be adapted to centre the exoskeleton 3 and the brace 100 over the particular part of the body, for example the olecranon 400, even after repeated flexion and extension of the joint. The exoskeleton 3 may comprise more or less than two loops to act as a guide 15. The guides 15 may be operable to retain the strap 13 over the part of the body, for example the olecranon 400 or patella. In some configurations, the exoskeleton 3 may not have a guide 15.

The properties of the strap 13, the placement of the anchors 11, 11′ on a wearer's arm, the length of the sleeve of the baselayer 1 and the placement of the fasteners 27, 29 on the sleeve may all or individually control the selected range of movement of the joint. For example, in some configurations the fasteners 27, 29 of the strap 13 are detachable and reattachable from one or both of the anchors 11, 11′. For example, one of the fasteners 27, 29 may comprise hook and loop material. Because the fasteners 27, 29 are detachable and reattachable they are operable to detachably fasten the strap 13 to the anchors 11, 11′ so that the strap 13 is releasably reattachable. Because the strap 13 comprises two fasteners 27, 29, either one fastener 27, 29 may be detached or both fasteners 27, 29 may be detached, so that the strap 13 no longer inhibits the range of movement of the joint. Therefore the whole strap 13 is detachable from the brace 100. If one or both fasteners 27, 29 are detached, the selected range of movement of the joint is adjustable.

In some configurations the strap 13 is coupled to the baselayer 1 by stitching or adhesive. In some configurations the strap 13 runs over the baselayer 1, or inside the baselayer 1. In some configurations, the strap 13 does not slide over the exoskeleton 3. In some configurations the strap 13 is covered in a protective layer to protect the strap from being accidentally knocked or moved and to improve the aesthetics of the brace 100. The strap 13 may have a cross-section adapted to ensure comfort with the wearer, for example the strap 13 may have an oval or rounded cross-section, or take any other regular or irregular shape.

For the example strap 13 shown in FIGS. 6a to 8c, in some configurations the tube 25 of the strap 13 comprises a cross-section different to that of the core 23. For example the tube 25 may have a channel with a complementary cross-section to the core 23 but a different cross-section for the outer part of the tube 25. For example, the cross-section of the outer part of the tube 25 may comprise oval or have rounded edges, so that it is more comfortable for a user wearing the brace 100. The cross-section of the core 23, the tube 25 and the channel 35 may take any regular or irregular shape.

In some configurations the strap 13 is biased to return to a selected position. In some configurations the strap 13 comprises a biasing means, for example an elasticated band, a resiliently deformable member, a magneto resistive element or a spring, for example a hair spring. In some configurations the core 23 comprises a biasing means, for example the core may comprise an elasticated band, a resiliently deformable member, a magneto resistive element or a spring, for example a hair spring. In some configurations the tube 25 comprises a biasing means, for example the tube may comprise an elasticated band, a resiliently deformable member, a magneto resistive element or a spring, for example a hair spring. In some configurations the strap 13 is resiliently deformable. In some configurations the strap may be flexible but not stretchable.

In some configurations the strap 13 may be located on the inside of the joint, for example so that tension in the strap limits extension of the joint, or so that compression in the strap limits flexion of the joint. In some configurations the strap 13 is adapted to only inhibit flexion of the joint. In some configurations the strap 13 is adapted to only inhibit extension of the joint.

In some configurations the strap 13 comprises a biasing means coupled to the core 23 running within the tube 25. The biasing means may inhibit the range of movement of the joint. The degree of resistance of the biasing means inhibiting the range of movement of the joint may be defined by a first coefficient of resistance (for example force per unit displacement). The biasing means may be coupled to the core 23 and the tube 25, or to the core 23 and one of the fasteners 27, 29. In this way, the strap 13 may be configured such that the core 23 is biased to return to a first position. This may prevent the core 23 from kinking or getting stuck within the tube 25.

In some configurations, the strap 13 provides an increased resistance, defined by a second coefficient of resistance, to inhibit movement of the joint over a second selected range of movement of the joint outside the first range. In some configurations the strap provides an even greater resistance than that defined by the second coefficient of resistance, defined by a third coefficient of resistance, to inhibit movement of the joint over a third selected range of movement of the joint outside the first and second ranges.

In some configurations the biasing means is used to provide resistance to movement of the joint over a selected range. This may be advantageous where bed-bound users may wish to exercise the joint.

In some configurations the core 23 may be flexible but not extensible. For example, the core 23 may not be stretchable. In some configurations the tube 25 may be flexible but not extensible. For example, the tube 25 may not be stretchable.

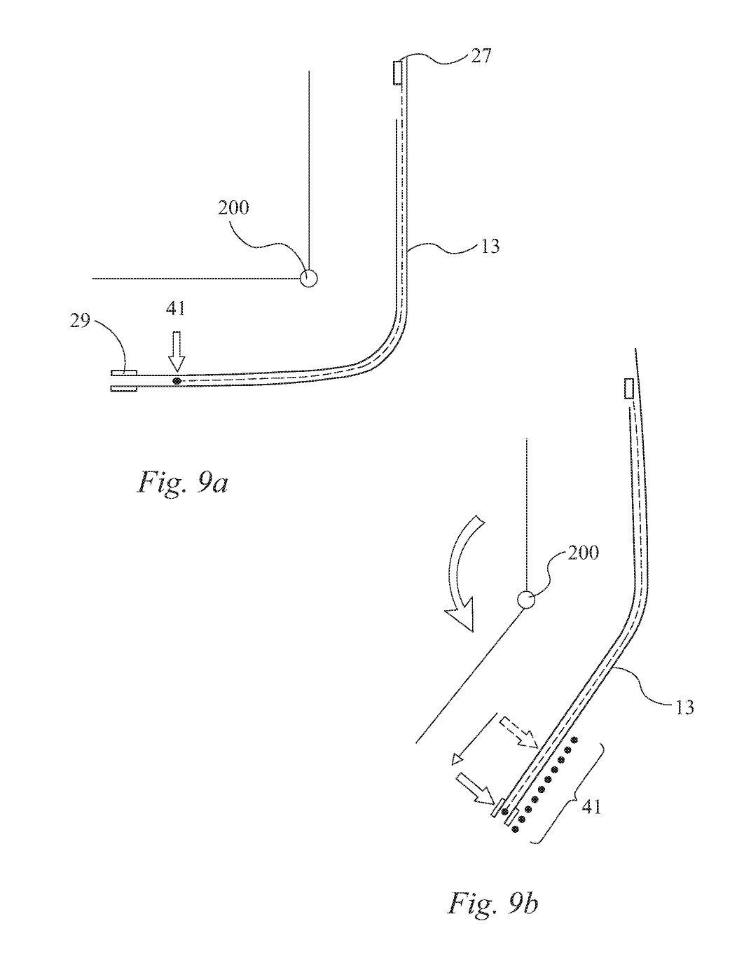

In some configurations the strap 13 comprises a movement detector to detect the degree of extension of the strap 13. For example, the movement detector may detect a degree of strain on the strap 13 or a degree of movement of the core 23 within the tube 25. The movement detector may comprise a sensor 41 as shown in FIG. 9a or a plurality of sensors as shown in FIG. 9b. The sensors 41 may be positioned within the tube 25 at one end of the core 23. In some configurations the movement detector may be one or more accelerometers. The incorporation of a movement detector would enable the brace 100 to be operable as an intelligent brace that could provide feedback regarding the use of the joint and be used to measure rehabilitation of a joint.

For the example strap 13 shown in FIGS. 6a to 8c, in some configurations the core 23 comprises more than one stop 31 or no stop 31. The stop 31 may take any regular or irregular shape. In some configurations the stop 31 is a lateral protrusion from the core 23. In some configurations the strap 13 may have more than one stop 31. For example, the strap 13 may have a stop 31 that is a vertical protrusion and a stop 31 that is a lateral protrusion. The tube 25 may comprise more than two retaining members 33 or no retaining member 33. The retaining members 33 may not be straps, for example, the retaining members 33 may be a bar. The retaining members 33 may be identical, or the retaining members may be different. The retaining members 33 may not be U-shaped. For example, the retaining members 33 may take any other regular or irregular shape.

The retaining members may not be adapted to fit over a portion of the tube 25. For example, the retaining members 33 may be adapted to lie over a portion of the channel 35. In some configurations the tube 25 does not comprise any notches 39. The tube 25 may comprise a plurality of grooves or ridges or other features adapted to interact with the retaining members 33. The tube 25 may comprise notches 39 only on one side. In some configurations the tube 25 does not comprise any slits 37. In some configurations the tube 25 is adapted to comprise slits 37 on a side facing away from the limb. In some configurations the tube 25 only comprises slits 37 over a selected portion of the tube, for example over a selected portion corresponding to a joint of the body.

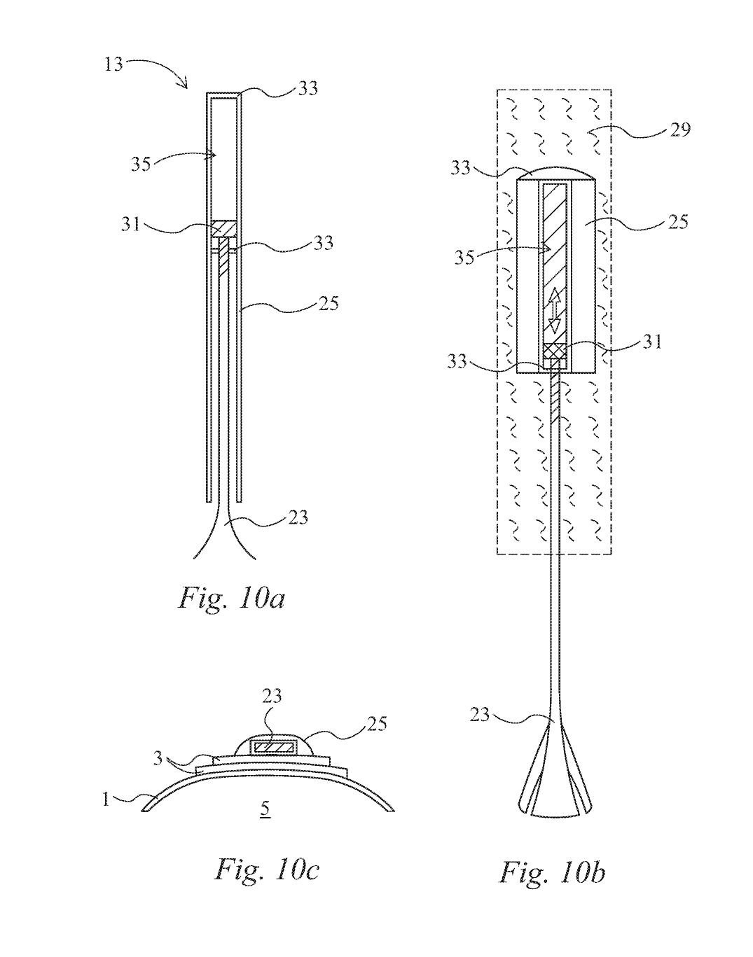

FIGS. 10a to 10c show an alternative configuration of a stop 31 and retaining member 33 arrangement for inhibiting the range of movement of a joint of the body. In this configuration, the stop 31 is a plunger at one end of the core 23 of the tube 25. The core 23 is narrower than the channel 35 of the tube 25, whereas the plunger fits snugly within the channel 35 so that the cross-section of the channel 35 is complementary to the cross-section of the plunger. The core 23 may splay slightly at the end opposite to the plunger. This may improve the coupling between the core 23 and the anchors 11, 11′. In this configuration, the placement of the retaining members 33 are not adjustable, and the retaining members 33 form part of the channel 35 of the tube 25.

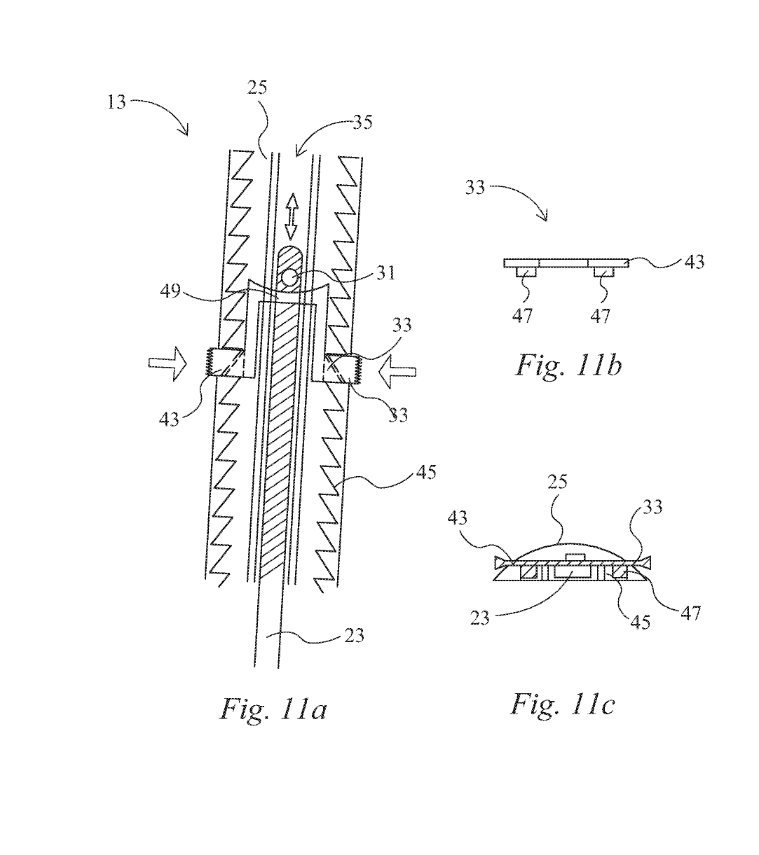

FIGS. 11a to 11c show an alternative configuration for the retaining members 33. The retaining members 33 are adapted to fit over and around a portion of the tube 25. However, here the retaining members 33 have two opposing push tabs 43 that are biased to engage with the tube 25 to provide an interference fit. The tube 25 further has a plurality of teeth 45 either side of the channel 35 that are engageable with respective teeth 47 on the retaining member 33. The retaining members 33 comprise a curved portion 49 between the teeth 45 and the push tabs 43 that is configured to abut with the stop 31 on the core 23.

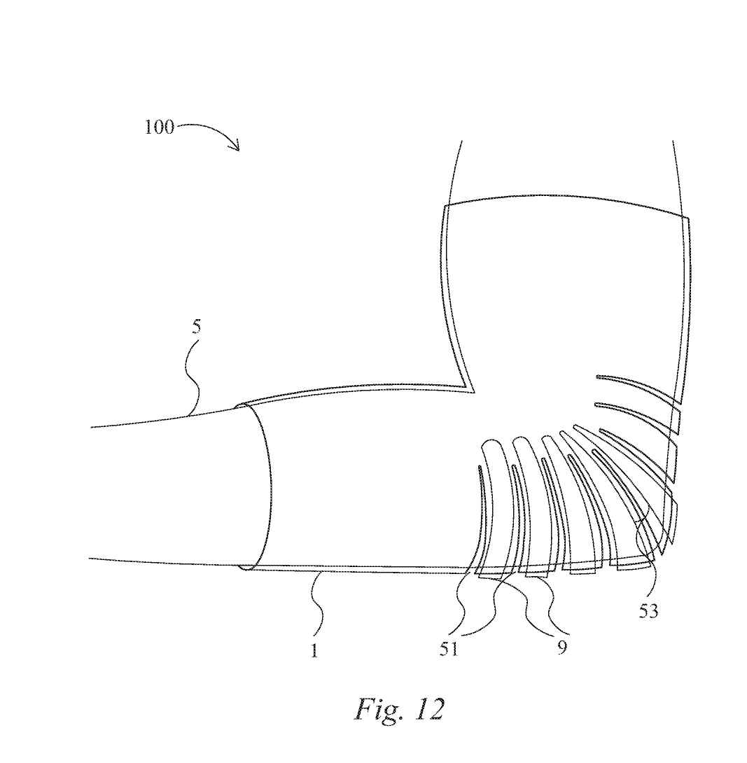

FIG. 12 shows an alternative configuration for the brace 100. Here the exoskeleton 3 comprises a plurality of separate ribs 9. The ribs 9 are sewn into the baselayer 1, however in some configurations the ribs 9 may be inserted into pockets in the baselayer 1 or may be coupled to the baselayer 1 by any other means. In this way, the ribs 9 may be arranged to be transverse to the nerve 7 to bridge across the nerve 7 when worn by the user. The baselayer 1 comprises slits 51 between the placement of the ribs 9 to improve flexibility and breathability of the brace 100 although the slits 51 may not be present in some configurations. The ribs 9 are coupled by a hinge 53, and in some configurations the ribs 9 are coupled by a living hinge although in other configurations the ribs 9 are separate and not coupled by a hinge. The living hinge may comprise a flexure bearing or a thinning of the material it connects.

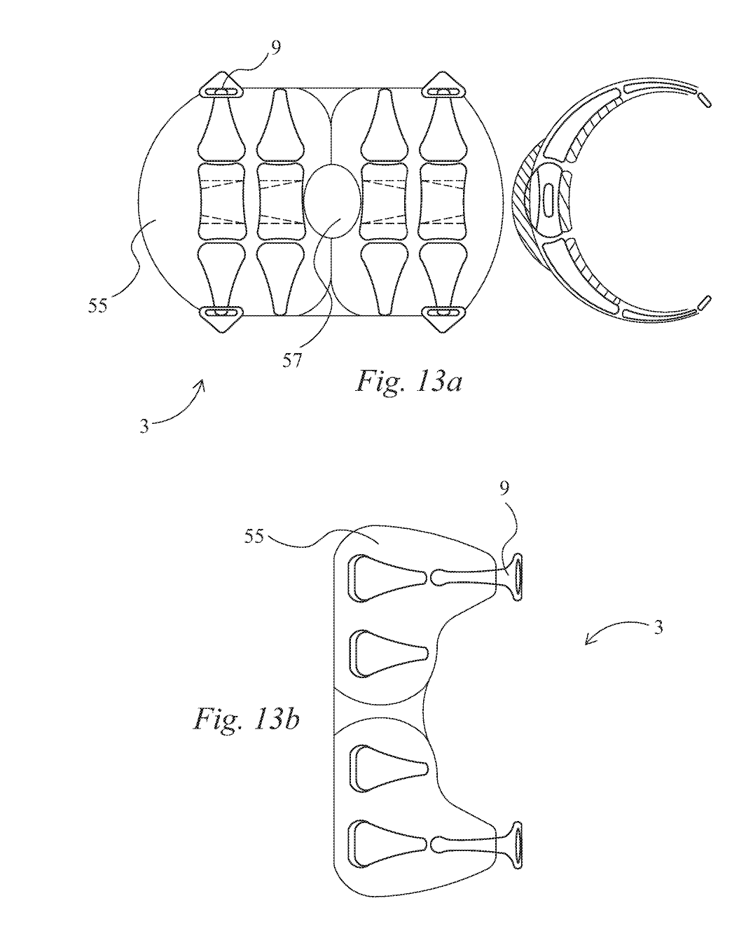

FIGS. 13a and 13b show an alternative configuration for the exoskeleton 3 for use with the brace 100. The exoskeleton 3 comprises four ribs 9, but may comprise more or less ribs 9 in other configurations. The ribs 9 are curved so that they are adapted to fit around a part of the body. The proximal and distal ribs 9 (i.e. the ribs 9 at either end of the exoskeleton 3) are larger than the other ribs 9 such that they are adapted to fit around a greater portion of the relevant part of the body. In this way, the ribs 9 may be arranged to cross the nerve 7 to bridge across the nerve 7 when worn by the user. The ribs 9 may be coupled by a flexible spine 55. The flexible spine 55 may comprise a hole or indentation 57 adapted to fit over a part of the body, for example the olecranon 400.

In some configurations the strap 13 is powered. For example the strap 13 may have a driving mechanism that is operable to slide the core 23 within the tube 25. In this way, the strap is operable to move the joint mechanically.

In some configurations the brace 100 may comprise a resilient member. For example, a brace apparatus can be provided to inhibit the range of movement of a joint of the body, in which the apparatus comprises a sleeve adapted to provide two anchors, one located either side of the joint; and a resilient member configured to be couplable to at least one of the two anchors such that when coupled to both of the anchors, the resilient member inhibits both flexion and extension of the joint, and when uncoupled from one of the anchors the resilient member inhibits flexion but not extension of the joint. This can enable a wearer easily to adapt the range of movement available to them, without completely losing the protection provided by the brace. For example the resilient member comprises a wedge, which may for example be provided by a compressible block of foam, as another example the resilient member comprises a biased hinge.

The resilient member may be located on the inside of the joint. The resilient member may be compressible, and may have a fixed degree of compressibility. For example, the resilient member may comprise a biasing means, for example an elasticated band, a resiliently deformable member, a magneto resistive element or a spring, for example a hair spring, inside a tube. The resilient member may inhibit movement of the joint, so that tension and/or compression inhibits extension and/or flexion of the joint. In some configurations the resilient member may comprise a wedge, for example a foam wedge. In some configurations the brace 100 comprises two resilient members, for example a resilient member on the inside of the joint and another resilient member on the outside of the joint. In some configurations the resilient member is adapted to only inhibit flexion of the joint. In some configurations the resilient member is adapted to only inhibit extension of the joint.

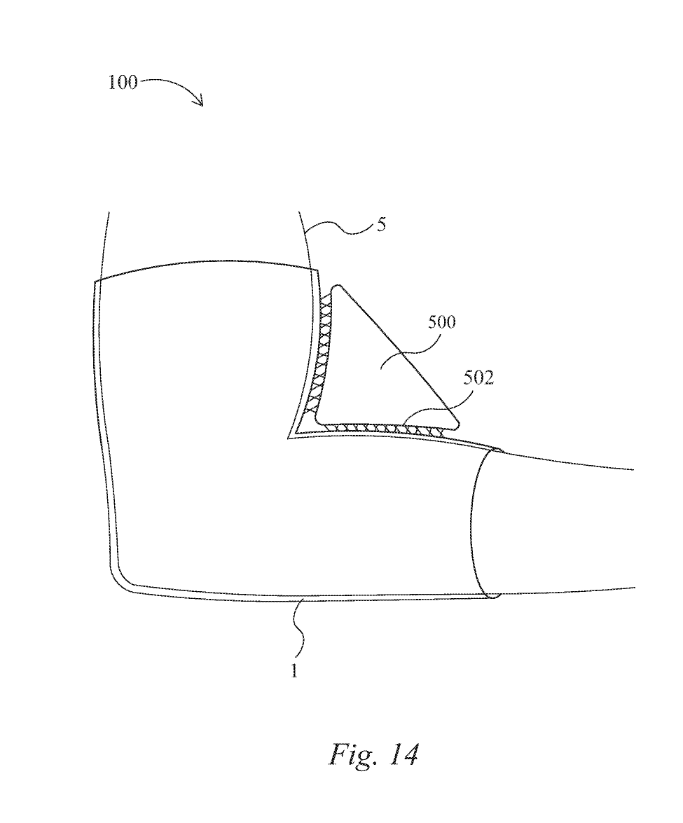

In some configurations, the resilient member may comprise a wedge 500, as shown in FIG. 14. The wedge 500 shown in FIG. 14 is on the inside of the joint, and may comprise a reattachable fastener 502 to releasably couple the wedge 500 to the exoskeleton 3 and/or the brace 100. In this way, the wedge 500 may inhibit movement of the joint, in this case flexion, outside a selected range more than within a selected ranged. In some configurations the wedge 500 may comprise a compressible material, for example foam. In some configurations the wedge 500 may be part of the exoskeleton 3. For example the ribs 9 of the exoskeleton 3 may extend from the outside of the joint to the inside of the joint.

In the context of the present disclosure other examples and variations of the apparatus and methods described herein will be apparent to a person of skill in the art.

1. A medical or surgical brace apparatus for treating a neuropathy and protecting the course of a nerve in a limb of the body, comprising:
a sleeve baselayer formed to fit, or configured to conform to fit, a portion of the limb around a joint and to grip the limb, such that a portion of the sleeve baselayer is proximal to the joint and a portion is distal of the joint; and
a deformable exoskeleton configured to fit around the joint of a body such that a portion of the exoskeleton is proximal to the joint and a portion is distal of the joint, the exoskeleton comprising a spine and a plurality of ribs coupled to the spine; wherein
the exoskeleton is coupled to the sleeve baselayer such that when the sleeve baselayer is worn fitted on the limb, the spine of the exoskeleton is aligned with, and arranged to follow, at least part of the course of the nerve, and the ribs are aligned transverse to the course of the nerve to bridge across the nerve to protect it.

2. The apparatus of claim 1 wherein the apparatus is configured to treat an ulnar neuropathy, and the sleeve baselayer and exoskeleton are formed to fit, or configured to conform to fit, around an elbow joint.

3. The apparatus of claim 1 or 2 comprising a flexible strap configured to be couplable to the baselayer to inhibit movement of the joint outside a selected range more than within the selected range.

4. The apparatus of any claim 3 wherein the exoskeleton comprises a guide to limit movement of the strap in a direction transverse to the longitudinal axis of the limb.

5. A medical or surgical brace apparatus for protecting the course of a nerve in a limb of the body, comprising:
a baselayer; and
a deformable exoskeleton;
wherein the baselayer is formed to fit the limb; and
the exoskeleton is coupled to the baselayer such that when the baselayer is worn fitted on the limb, the exoskeleton is aligned with, and arranged to follow at least a part of the course of the nerve and to bridge across the nerve to protect it.

6. The apparatus of claim 5 wherein the exoskeleton is arranged to bridge across the nerve at at least two separate places along its course

7. The apparatus of claim 5 or 6, in which the exoskeleton comprises an elongate spine arranged so that, when the base layer is worn fitted to the limb, the course of the spine corresponds to the course of the nerve.

8. A medical or surgical brace apparatus for protecting a region of a limb of the body, comprising:
a baselayer; and
a deformable exoskeleton; wherein
the baselayer is formed to fit the limb; and
the exoskeleton is coupled to the baselayer such that when the baselayer is worn fitted on the limb the exoskeleton is arranged to bridge across the region of the limb at at least two separate places.

9. The apparatus of claim 5, 6, or 7 wherein the nerve is the ulnar nerve, or the apparatus of claim 8 wherein the region of the limb corresponds to the course of the ulnar nerve.

10. The apparatus of any one of claims 5 to 9 wherein the baselayer comprises a sleeve adapted to fit to a first portion of a limb, proximal to a joint, and a second part of the limb, distal to the joint, wherein the region of the limb, or the course of the nerve, extends across the joint.

11. The apparatus of any one of claims 5 to 10 wherein the exoskeleton bridges across the region of the limb or the course of the nerve such that, upon the application of pressure to the exoskeleton, pressure is distributed either side of the region of the limb or nerve.

12. The apparatus of any one of claims 5 to 11 wherein, to bridge across the region of the limb or nerve in two separate places, the exoskeleton comprises at least two ribs, each arranged to bridge across the region of the limb or nerve.

13. The apparatus of claim 12 wherein the ribs are coupled together at their ends.

14. The apparatus of claim 13 wherein the ribs are coupled to form a meandering pattern.

15. A medical or surgical apparatus for inhibiting the range of movement of a joint of the body, comprising:
a sleeve adapted to wrap around a limb either side of the joint of the body, wherein the sleeve provides two anchors, one located either side of the joint; and
a flexible strap configured to be couplable to the two anchors;
such that when coupled to the anchors, the strap inhibits movement of the joint outside a selected range, more than within the selected range.

16. The apparatus of claim 15 adapted to protect a region of the limb and comprising an exoskeleton coupled to the sleeve such that when the sleeve is worn on the limb the exoskeleton is arranged to bridge across the region of the limb at least twice.

17. The apparatus of claim 5 or 8 wherein the exoskeleton comprises at least two ribs.

18. The apparatus of any one of claims 15 to 17 comprising a resilient member adapted to fit on the inside of the joint and inhibit movement of the joint outside a selected range.

19. The apparatus of any one of claims 5 to 14 comprising a flexible strap configured to be couplable to the baselayer to inhibit movement of the joint outside a selected range more than within the selected range.

20. The apparatus of any of claims 15 to 19 wherein the strap is extendible.

21. The apparatus of claim 20 wherein the strap comprises a biasing means to inhibit extension of the strap.

22. The apparatus of any of claims 15 to 21 wherein the strap has a fixed maximum length.

23. The apparatus of any of claims 15 to 22 wherein the strap comprises a reattachable fastener, the fastener being releasable such that the strap no longer inhibits the range of movement of the joint.

24. The apparatus of claim 23 wherein the fastener is arranged to couple the strap to at least one of the anchors.

25. The apparatus of any one of claims 15 to 24 wherein the strap comprises a core arranged to be slidable within a tube within a range corresponding to the selected range of movement of the joint.

26. The apparatus of claim 25 wherein the tube is configured to be coupled to one anchor and the core is configured to be coupled to the other anchor.

27. The apparatus of claim 25 or 26 comprising a driving mechanism to slide the core within the tube to mechanically move the joint.

28. The apparatus of any one of claims 15 to 27 comprising a movement detector to detect extension of the strap.

29. The apparatus of claim 28 wherein the movement detector detects strain on the strap.

30. The apparatus of claim 29 as dependent on any one of claims 25 to 27 wherein the movement detector detects a degree of movement of the core within the tube.

31. The apparatus of any one of claims 15 to 30 comprising a guide to limit movement of the strap in a direction transverse to the longitudinal axis of the limb.

32. The apparatus of claim 31 wherein the guide is adapted to position at least a portion of the strap over the bony prominence at the outer edge of the joint.

33. The apparatus of any one of claims 15 to 32 as dependent upon any of claims 9 to 14 in which at least one of the ribs is arranged distal to the joint.

34. The apparatus of any one of claims 15 to 33 as dependent upon any of claims 9 to 14 in which at least one of the ribs is arranged proximal to the joint.

35. The apparatus of claim 32 or 33 as dependent upon claim 30 or 31 wherein the guide is coupled to the exoskeleton.

36. A brace according to any one of claims 5 to 14 comprising an apparatus according to any one of claims 15 to 35, wherein the baselayer comprises the sleeve.

37. A brace according to any one of claims 5 to 14 comprising an apparatus according to claim 31 or 32 wherein the exoskeleton comprises the guide.

38. A medical or surgical apparatus for inhibiting the range of movement of a joint of the body, comprising:
a sleeve adapted to provide two anchors, one located either side of the joint; and
a resilient member configured to be couplable to at least one of the two anchors such that when coupled to both of the anchors, the resilient member inhibits both flexion and extension of the joint, and when uncoupled from one of the anchors the resilient member inhibits flexion but not extension of the joint.

39. The apparatus of claim 38 wherein the resilient member comprises a wedge.

40. The apparatus of claim 39 wherein the wedge is provided by a compressible block of foam.

41. The apparatus of claim 39 wherein the resilient member comprises a biased hinge.

42. A medical or surgical apparatus for controlling movement of a joint of the body, comprising:
a sleeve adapted to wrap around a limb either side of the joint of the body, wherein the sleeve provides two anchors, one located either side of the joint; and
a strap configured to be couplable to the two anchors;
a driver coupled to at least one end of the strap;
such that when operated, the driver controls the strap to move the joint.

43. The apparatus of claim 42 wherein the strap comprises a core slidable within a tube, wherein operation of the driver controls movement of the core within the tube.

44. The apparatus of claim 42 or 43 adapted to protect a region of the limb and comprising an exoskeleton coupled to the sleeve such that when the sleeve is worn on the limb the exoskeleton is arranged to bridge across the region of the limb at least twice

45. A kit of parts for a medical or surgical brace apparatus for treating a neuropathy and protecting the course of a nerve in a limb of the body, comprising:
a sleeve baselayer formed to or configured to conform to a portion of the limb around a joint and to grip the limb, such that a portion of the sleeve baselayer is proximal to the joint and a portion is distal of the joint; and
a deformable exoskeleton configured to fit around the joint of a body such that a portion of the exoskeleton is proximal to the joint and a portion is distal of the joint, the exoskeleton comprising a spine aligned with the course of the nerve and a plurality of ribs coupled to the spine and extending transverse to the course of the nerve; wherein
the exoskeleton is couplable to the sleeve baselayer such that when the sleeve baselayer is worn fitted on the limb and the exoskeleton is coupled to the sleeve baselayer, the exoskeleton is aligned with, and arranged to follow at least part of the course of the nerve to bridge across the nerve to protect it.

46. A kit of parts for a medical or surgical brace according to any one of the preceding claims.

47. An exoskeleton for a kit of parts according to claim 45 or 46.

48. A medical or surgical brace substantially as described herein with reference to the accompanying drawings.

49. A medical or surgical apparatus substantially as described herein with reference to the accompanying drawings.
